(19) United States
(12) Patent Application Publication
(10) Pub. No.: US 2024/0108885 A1
(43) Pub. Date: Apr. 4, 2024

(54) FIXATION COMPONENTS FOR IMPLANTABLE MEDICAL DEVICES

(71) Applicant: Medtronic, Inc., Minneapolis, MN (US)

(72) Inventors: Xin Chen, West Haven, CT (US); Vladimir Grubac, Brooklyn Park, MN (US); Brian P. Colin, Anoka, MN (US); Kathryn E. Hilpisch, Cottage Grove, MN (US); Michael D. Eggen, Chisago City, MN (US)

(21) Appl. No.: 18/469,331

(22) Filed: Sep. 18, 2023

Related U.S. Application Data
(63) Continuation of application No. 16/825,143, filed on Mar. 20, 2020, now Pat. No. 11,759,632.
(60) Provisional application No. 62/825,233, filed on Mar. 28, 2019.

(51) Int. Cl. A61N 1/05; B21D 39/02

(52) U.S. Cl. CPC A61N 1/059; B21D 39/02

(57) ABSTRACT

An example fixation component for an implantable medical device (IMD) includes a base and a plurality of tines configured be deployed with a target deployment stiffness to engage tissue a target implant site while maintaining a target deflection stiffness after deployment. The base defines a longitudinal axis of the fixation component and is fixedly attached near the distal end of the IMD. Each tine is spaced apart from one another around a perimeter of the distal end of the IMD and extend from the base. A shape of each tine is selected to control each of the target deployment stiffness and target deflection stiffness.

This application is a continuation of U.S. application Ser. No. 16/825,143, filed Mar. 20, 2020, which claims benefit of U.S. Provisional Patent Application No. 62/825,233, filed Mar. 28, 2019, both of which are incorporated herein by reference in their entirety.

### TECHNICAL FIELD

This disclosure is related to medical device systems, such as relatively compact implantable medical devices and associated fixation components.

### BACKGROUND

In some examples, implantable cardiac pacemakers include a pulse generator device to which one or more flexible elongate lead wires are coupled. The pulse generator device may be implanted in a subcutaneous pocket, remote from the heart, and each of the one or more lead wires extends therefrom to a corresponding electrode, coupled thereto and positioned at a pacing site, either endocardial or epicardial. Mechanical and/or MRI compatibility issues may be associated with elongate lead wires. Relatively compact implantable medical devices (IMDs) have been developed that are wholly contained within a relatively compact package, the entirety of which is configured for implant in close proximity to the pacing site, e.g., within a chamber of the heart.

### SUMMARY

This disclosure describes IMD fixation components having improved flexibility, fixation, or both to facilitate implanting IMDs, including relatively compact IMDs. A fixation component of an IMD may include a plurality of tines. Each respective tine of the plurality of tines has a deployment stiffness that enables the respective tine to penetrate the tissue at a target implant site. By controlling the deployment stiffness, the plurality of tines may have improved tissue fixation, including, for example, controlling of a depth of tine penetration and an amount of tissue engagement in a lateral direction. Each respective tine of the plurality of tines also has a deflection stiffness that may enable a clinician to confirm adequate fixation of the tines into the tissue of the patient. For example, a pull test or tug test may be performed under fluoroscopy to confirm that the plurality of tines have engaged the tissue. By controlling the deflection stiffness, the plurality of tines may have an improved flexibility that enables a clinician to more easily confirm tissue engagement.

In some examples, a fixation component for an implantable medical device (IMD) may include a base defining a longitudinal axis of the fixation component and a plurality of tines extending from the base and being spaced apart from one another. The base may be fixedly attached to the IMD having a proximal end and a distal end aligned along the longitudinal axis. Each tine of the plurality of tines may include a proximal portion and a distal portion. The Proximal portion may include a proximal section fixedly attached to the base and extending from the base in a first direction; a first curved section defining a first deformable pre-formed curvature and extending from the proximal section laterally, outward from the longitudinal axis; a first straight section extending from the first curved section laterally, outward from the longitudinal axis in a second direction; and a second curved section defining a second deformable pre-formed curvature and extending from the first straight section laterally, outward from the longitudinal axis. The distal portion may include a second straight section extending from the second curved section in a third direction oriented generally opposite the first direction; a third curved section defining a third deformable pre-formed curvature and extending from the second straight section; and tip section extending from the third curved section toward the longitudinal axis and terminating in a free distal end.

In some examples, a fixation component for an implantable medical device (IMD), may include a base defining a longitudinal axis of the fixation component and a plurality of tines extending from the base and being spaced apart from one another. The base may be fixedly attached to the IMD having a proximal end and a distal end aligned along the longitudinal axis. Each tine of the plurality of tines may include a proximal portion and a distal portion. The proximal portion may include a proximal section fixedly attached to the base and extending in a first direction generally parallel to the longitudinal axis; and a first curved section extending from the proximal section laterally, outward from the longitudinal axis, wherein the curved section is configured to provide a deflection stiffness of less than about 0.6 Newtons. The distal portion may include a second proximal section extending from the first curved section in a second direction oriented generally opposite the first direction; a second curved section having a deformable pre-formed curve and extending from the second proximal section; and tip section extending from the second curved section toward the longitudinal axis and terminating in a free distal end.

In some examples, an implantable medical device (IMD) may include a housing extending along a longitudinal axis from a proximal end to a distal end; an electrode mounted in proximity to the distal end of the housing; and a fixation component. The fixation component may include a base in proximity to the distal end of the housing and a plurality of tines fixedly attached spaced from one another around a perimeter of the distal end of the housing. Each tine of the plurality of tines may include a proximal portion and a distal portion. The proximal portion may include a proximal section fixedly attached to the base and extending from the base in a first direction; a first curved section defining a first deformable pre-formed curvature and extending from the proximal section laterally, outward from the longitudinal axis; a first straight section extending from the first curved section laterally, outward from the longitudinal axis in a second direction; and a second curved section defining a second deformable pre-formed curvature and extending from the first straight section laterally, outward from the longitudinal axis. The distal portion may include a second straight section extending from the second curved section in a third direction oriented generally opposite the first direction; a third curved section defining a third deformable pre-formed curvature and extending from the second straight section; and tip section extending from the third curved section toward the longitudinal axis and terminating in a free distal end.

In some examples, a medical device system may include an implantable medical device (IMD) including a housing extending along a longitudinal axis from a proximal end to a distal end; an electrode mounted in proximity to the distal end of the housing; and a fixation component that includes a base in proximity to the distal end of the housing and a plurality of tines fixedly attached spaced from one another around a perimeter of the distal end of the housing; and a delivery tool including a tubular sidewall that defines a lumen into which the IMD may be loaded, wherein the lumen having a distal opening through which the IMD may be deployed. Each tine of the plurality of tines including a proximal portion that includes a proximal section fixedly attached to the base and extending from the base in a first direction; a first curved section defining a first deformable pre-formed curvature and extending from the proximal section laterally, outward from the longitudinal axis; a first straight section extending from the first curved section laterally, outward from the longitudinal axis in a second direction; and a second curved section defining a second deformable pre-formed curvature and extending from the first straight section laterally, outward from the longitudinal axis; and a distal portion that includes a second straight section extending from the second curved section in a third direction oriented generally opposite the first direction; a third curved section defining a third deformable pre-formed curvature and extending from the second straight section; and tip section extending from the third curved section toward the longitudinal axis and terminating in a free distal end.

In some examples, a method of forming a fixation component for an IMD may include forming a base defining a longitudinal axis of the fixation component; and forming a plurality of tines extending from the base and being spaced apart from one another. Each tine of the plurality of tines may include a proximal portion including a proximal section fixedly attached to the base and extending from the base in a first direction; a first curved section defining a first deformable pre-formed curvature and extending from the proximal section laterally, outward from the longitudinal axis; a first straight section extending from the first curved section laterally, outward from the longitudinal axis in a second direction; and a second curved section defining a second deformable pre-formed curvature and extending from the first straight section laterally, outward from the longitudinal axis; and a distal portion including a second straight section extending from the second curved section in a third direction oriented generally opposite the first direction; a third curved section defining a third deformable pre-formed curvature and extending from the second straight section; and tip section extending from the third curved section toward the longitudinal axis and terminating in a free distal end.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating a portion of an example medical device system configured to implant a relatively compact IMD at a target implant site.

FIG. 2A is a conceptual diagram illustrating a plan view of a relatively compact IMD including a fixation component.

FIG. 2B is a conceptual diagram illustrating the IMD of FIG. 2A implanted at a target implant site.

FIG. 3A is a conceptual diagram illustrating an elevation view of an example two-knuckle fixation component.

FIG. 3B is a conceptual diagram illustrating an end view of the two-knuckle fixation component of FIG. 3A.

FIGS. 4A-4E are conceptual diagrams illustrating plan views of a tine of the two-knuckle fixation component of FIGS. 3A and 3B, prior to forming curves in the tine.

FIGS. 5A and 5B are conceptual diagrams illustrating an example three-knuckle fixation component.

FIG. 6 is a conceptual diagram illustrating a plan view with a partial cut-away section of a medical device system including a delivery tool and an IMD.

FIG. 7A is a conceptual diagram illustrating a spring loaded configuration of a fixation component within a lumen of a delivery tool.

FIG. 7B is a conceptual diagram illustrating an initial release of the fixation component from the spring loaded configuration.

FIG. 7C is a conceptual diagram illustrating movement of the tines causing initial penetration of the tissue after the initial release of the fixation component.

FIG. 7D is a conceptual diagram illustrating further movement of the fixation component as the portions of the tines between the distal most and next proximal curves reaches the distal end of the delivery tool.

FIG. 7E is a conceptual diagram illustrating further movement fixation component as the proximal curve travels past the distal end of the delivery tool.

FIG. 7F is a conceptual diagram illustrating final configuration of fixation component movement, subsequent to movement.

FIG. 8 is a flow diagram illustrating an example method of manufacturing a fixation component.

### DETAILED DESCRIPTION

This disclosure describes IMD fixation components having improved flexibility, fixation, or both to facilitate implanting IMDs, such as relatively compact IMDs. An example fixation component for an IMD may include a base and a plurality of tines. The plurality of tines is configured be deployed with a target deployment stiffness to engage tissue a target implant site while maintaining a target deflection stiffness after deployment to enable visualization (e.g., via fluoroscopy) of engagement with the tissue. The base may define a longitudinal axis of the fixation component, e.g., a proximal end and a distal end of the IMD may be aligned along the longitudinal axis. The base may be fixedly attached to the IMD near the distal end of the IMD. The plurality of tines may be spaced apart from one another around a perimeter of the distal end of the IMD and extend from the base. A shape of each respective tine of the plurality of tines may be selected to control each of the target deployment stiffness and target deflection stiffness. For example, the shape of a respective tine may include a number of preformed curves on the respective tine, a curvature (e.g., radius) of each preformed curve on the respective tine, a length of each preformed curve, a length of straight sections between preformed curves, a width of the respective tine or sections thereof (e.g., one or more tapered portions), a thickness of the respective tine, a number of cutouts along the length of the respective tine, shapes of cutouts, or any combination thereof.

Each tine of the plurality of tines may include a proximal portion and a distal portion. The proximal portion may include a proximal section and at least one curved section. In some examples, the proximal portion may include a curved section. In some examples, the proximal portion may include a first curved section, a second curved section, and a first straight section between the first and second curved sections. The proximal section is fixedly attached to the base and extends from the base in a first direction. For example, the first direction may be substantially parallel (e.g., parallel or nearly parallel within the capabilities of fixation component manufacturing techniques) to the longitudinal axis or at some angle relative to the longitudinal axis. The first curved section may define a first deformable pre-formed curvature and extend from the proximal section laterally, outward from the longitudinal axis. The first straight section extends from the first curved section laterally, outward from the longitudinal axis in a second direction. The second curved section defines a second deformable pre-formed curvature and extends from the first straight section laterally, outward from the longitudinal axis. The distal portion may include a second straight section extending from the second curved section in a third direction oriented generally opposite the first direction, a third curved section defining a third deformable pre-formed curvature and extending from the second straight section, and tip section extending from the third curved section toward the longitudinal axis and terminating in a free distal end.

When deployed at a target implant site, the tines have a deployment stiffness that enables a respective tine to penetrate the tissue at a target implant site. By controlling the deployment stiffness, the tines may have improved tissue fixation, including control of a depth of tine penetration and an amount of tissue engagement in a lateral direction. After deployment at the target implant site, a deflection stiffness of the tines enables a clinician to confirm adequate fixation of the tines into tissue of a patient. For example, a pull test or tug test may be performed under fluoroscopy to confirm that the tines have engaged the tissue to confirm adequacy of implantation of the IMD. The pull test or tug test may include the clinician pulling or tugging on the deployed IMD and observing movement of the tines to determine if the tines are engaged in tissue, e.g., the tines that are embedded in tissue deflect or bend as deployed IMD is pulled or tugged. By controlling the deflection stiffness, the tines may have an improved flexibility that enables a clinician to more easily confirm tissue engagement.

In this disclosure, the example systems, devices, and techniques will be described with reference to delivering an IMD to a target site in a heart of a patient. However, it will be understood that example systems, devices, and techniques of the present disclosure are not limited to delivering IMDs to a target site in the heart. For example, example systems, devices, and techniques described herein may be used to deliver other medical devices, such as drug delivery device, sensing devices, neurostimulation device, or medical electrical leads to other locations within a body of a patient. In short, the example systems, devices, and techniques described herein can find useful application in delivery of a wide variety of implantable medical devices for delivery of therapy to a patient or patient sensing.

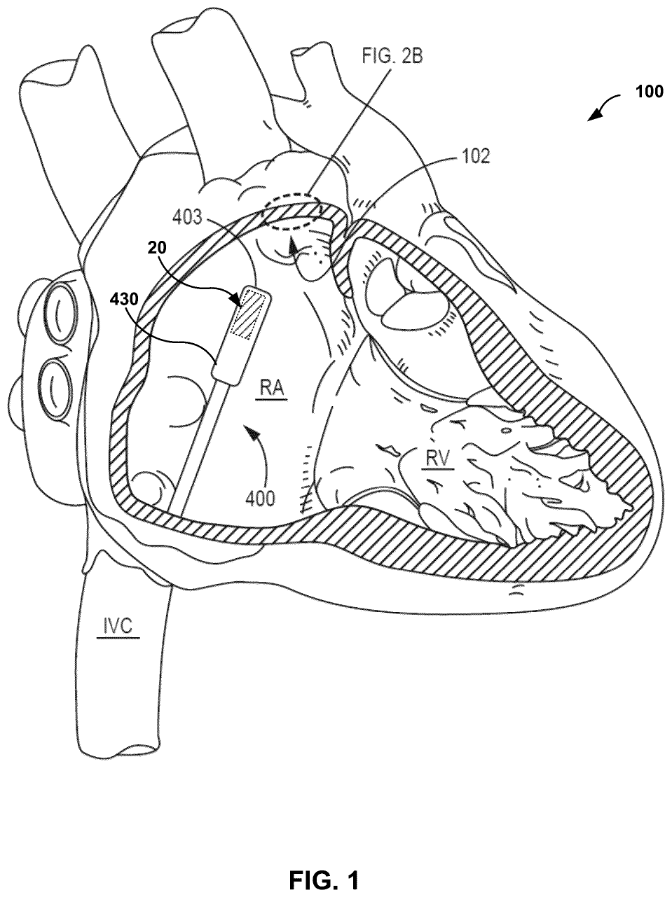

FIG. 1 is a conceptual diagram illustrating a portion of an example medical device system 400 configured to implant a relatively compact implantable medical device 20 (“IMID 20”) at a target implant site 102. In some examples, as illustrated in FIG. 1, the target implant site 102 may include an appendage of a right atrium RA of the heart 100 of a patient. In some examples, target implant site 102 may include other portions of heart 100 or other locations within a body of the patient. Medical device system 400 may include a delivery tool 430 configured to house and controllably deploy relatively compact IMD 20. In some examples, a clinician may maneuver medical device system 400 to target implant site 102. For example, with the IMD loaded therein, the clinician may guide delivery tool 430 up through the inferior vena cava IVC and into the right atrium RA. In some examples, other pathways or techniques may be used to guide delivery tool 430 into other target implant sites within the body of the patient.

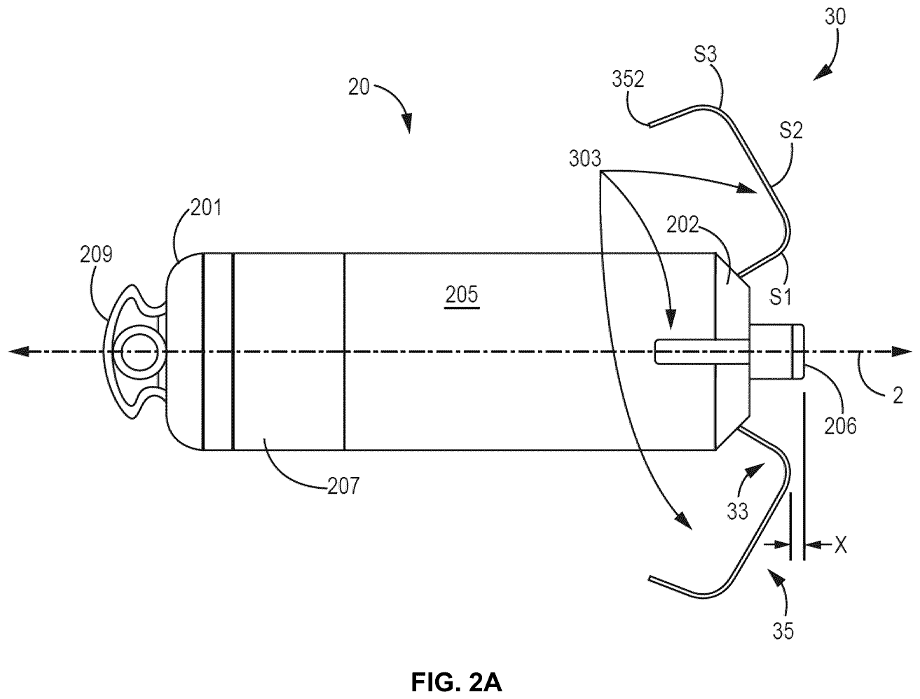

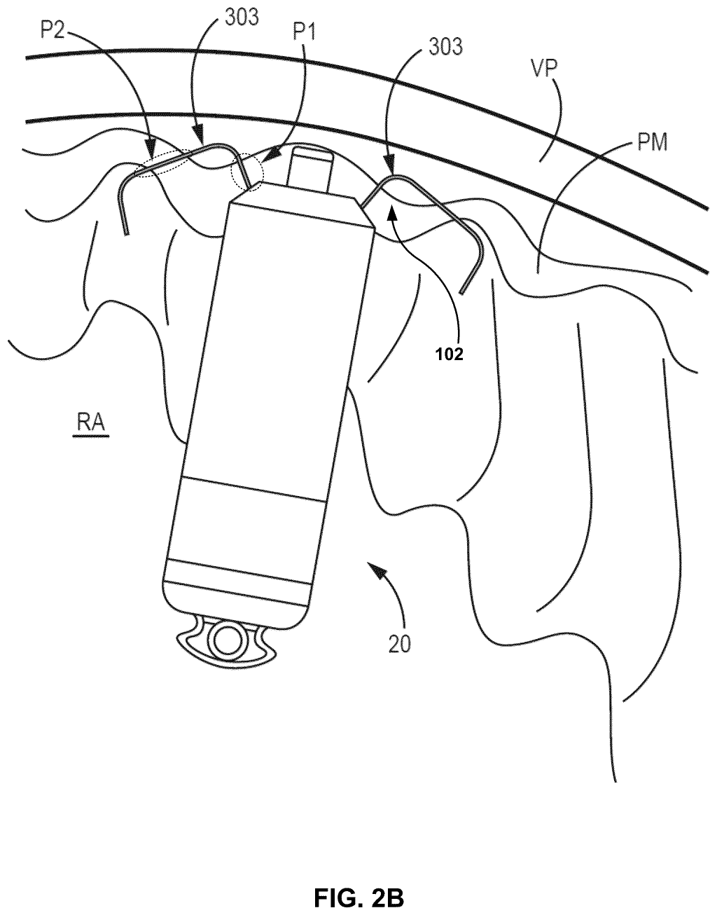

FIG. 2A is a conceptual diagram illustrating a plan view of a relatively compact IMD 20 including a fixation component 30. IMD 20 includes housing 205 extending along longitudinal axis 2 from a proximal end 201 to a distal end 202. Housing 205 may be formed from a biocompatible and biostable metal such as titanium. In some examples, housing 205 may include a hermetically sealed housing. IMD 20 may include any suitable dimensions. In some examples, an outer diameter of IMD 20 (e.g., outer diameter of housing 205) may be between about 10 French and about 30 French, such as about 20 French.

IMD 20 may contain electronic circuitry, including one or more of sensing circuitry (e.g., for sensing cardiac signals), therapy delivery circuitry (e.g., for generating cardiac pacing pulses), and processing circuitry for controlling the functionality of IMD 20, and may include an electrode 206. The electronic circuitry may be configured to generate and deliver an electrical pulse therapy to tissue proximate electrode 206. Electrode 206 may be spaced apart from distal end 202 of housing 205, for example, being coupled to the sensing and therapy delivery circuitry by a conductor of an hermetic feedthrough assembly (not shown). In some examples, IMD 20 includes a holding member 209 fixedly attached to proximal end 201 of housing 205, wherein holding member 209 is configured for temporarily tethering IMD 20 to a delivery tool, such as delivery tool 430. Housing 205 may be overlaid with an insulative layer, for example, medical grade polyurethane, parylene, or silicone. The insulative layer may define second electrode 207, for example, by removing a portion of the insulative layer to expose the metallic surface of housing 205. Electrode 206 may function in conjunction with second electrode 207 for bipolar pacing and sensing.

Fixation component 30 includes a plurality of tines 303 (“tines 303”). Tines 303 may be configured to hold electrode 206 in contact with tissue at a target implant site, e.g., target implant site 102. In some examples, electrode 206 may longitudinally be approximately flush with a distal-most portion of tines 303 (e.g., relative to longitudinal axis 2), or distal thereto by a distance “X” that may be up to about 2 millimeters (mm). Tines 303 include a proximal portion 33 and a distal portion 35. Each of proximal portion 33 and distal portion 35 may include one or more sections. For example, as illustrated in FIG. 2A, tines 303 may include first section S1, second section S2, and third section S3. In other examples, tines 303 may include fewer section, such as two sections, or more sections, such as more than three sections. Each of first, second, and third section S1, S2, and S3 may include an elastically deformable material pre-formed into a curved section and/or a substantially straight section. In the example illustrated in FIG. 2A, first section S1 is fixedly attached to distal end 202 of device housing 205 and extends around a pre-formed curvature to second section S2. Second section S2 extends proximally along a relatively straight line to third section S3. Third section S3 extending around a pre-formed curvature to a free distal end 352.

Tines 303 may be configured to have a target deflection stiffness and a target deployment stiffness. The target deflection stiffness may include a measure of a resistance to force applied to IMD 20 in a proximal direction when fixation component 30 is engaged with tissue at target site 102. In some examples, the target deflection stiffness may be selected to enable tines 303 to deflect a predetermined amount to enable visualization of tines 303 under fluoroscopy. In some examples, the target deflection stiffness may be within a range from about 0.2 N to about 0.8 N, such as about 0.3 N to about 0.6 N. The deployment stiffness may include a measure of a force applied by tines 303 as tines 303 move from a deformed configuration to an undeformed configuration upon deployment of fixation component 30 from distal opening 403 of delivery tool 430 (FIG. 1) such that free distal end 352 penetrates pectinate muscle PM. In some examples, the target deployment stiffness may be within a range from about 0.6N to about 1.2N.

FIG. 2B is a conceptual diagram illustrating IMD 20 implanted at target implant site 102. Target implant site 102 includes a portion the right atrial RA wall having a laminate structure that includes an inner layer of pectinate muscle PM and an outer layer of visceral pericardium VP, which forms the epicardial surface. IMD 20 is secured at target implant site 102 by tines 303 of fixation component 30 penetrating through the layer of pectinate muscle PM without perforating through visceral pericardium VP. Perforation of the visceral pericardium VP may result in pericardial effusion. Tines 303 are configured for spring-loaded release, upon deployment out through distal opening 403 of delivery tool 430 (FIG. 1) such that free distal end 352 penetrates pectinate muscle PM without perforating visceral pericardium VP. It should be noted that alternate suitable implant sites for embodiments of fixation member tines described herein can be along any endocardial surface defined by pectinate muscle PM.

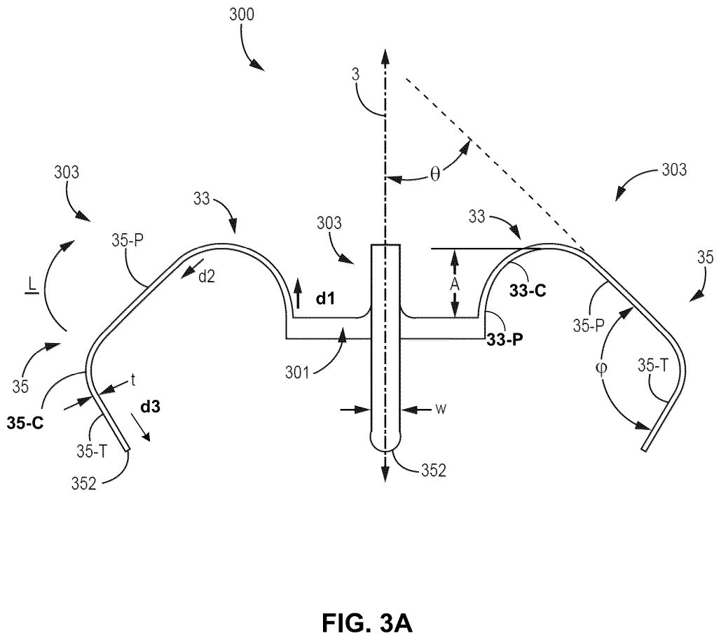

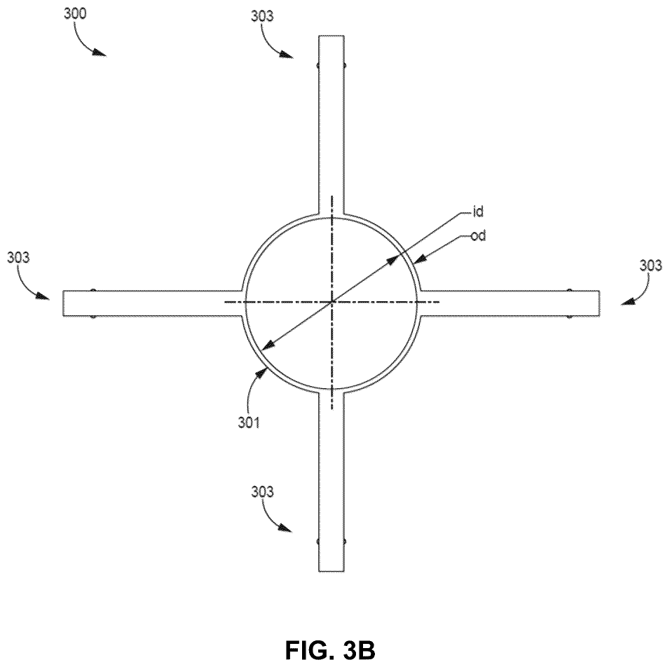

FIG. 3A is a conceptual diagram illustrating an elevation view of an example two-knuckle fixation component 300. FIG. 3B is a conceptual diagram illustrating an end view of fixation component 300 of FIG. 3A. Two-knuckle fixation component 300 may be the same as or substantially similar to fixation component 30, except for the differences described herein. For example, two-knuckle fixation component 300 includes tines 303. Tines 303 are illustrated in a relaxed configuration, e.g., a configuration without application of any external forces on any portion of tines 303. Fixation component 300 is referred to as a two-knuckle fixation component because it has two curved sections resulting two knuckles.

As illustrated in FIG. 3A, two-knuckle fixation component 300 includes a base 301 from which tines 303 extend. Base 301 may define a longitudinal axis 3 of two-knuckle fixation component 300. When base 301 is mounted around distal end 202 of device housing 205 such that a perimeter of two-knuckle fixation component 300 extends around electrode 206, longitudinal axis 3 is generally aligned along longitudinal axis 2 of IMD 20 (FIG. 2A).

As illustrated in FIG. 3B, tines 303 are spaced apart from one another around a perimeter of base 301. Base 301 may have an inner diameter (“id”) of about 0.20 inch (5.08 millimeters, mm) and an outer diameter od of about 0.21 inch (5.334 mm). In some examples, two-knuckle fixation component 300 may be mounted to distal end 202 of device housing 205, for example, in a manner the same or substantially similar to that described in commonly assigned U.S. Pat. No. 10,099,050B2 (filed on Jan. 19, 2017), which is incorporated herein by reference in its entirety. In some examples, two-knuckle fixation component 300 may include separately formed tines 303 that are individually mounted to distal end 202 of device housing 205 (e.g., not integrated together with base 301).

Tines 330 may include any suitable elastically deformable biocompatible material. In some examples, tines 303 may include a super-elastic material, such as, for example, a nickel-titanium alloy. For example, two-knuckle fixation component 300 may be cut from a medical grade nickel-titanium alloy tubing that conforms to the chemical, physical, mechanical, and metallurgical requirements of the ASTM F2063 standard, and has a wall thickness of about 0.005 inch (0.127 mm). In this way, tines 303 may be integrally formed with base 301 and each tine of tines 303 may have a constant thickness “t” of about 0.005 inch±0.001 inch (0.127 mm±0.0254 mm). In some examples, after cutting tines 303, tines 303 may be shaped into a preformed configuration by bending and holding tines 303, while heat treating according to methods known to those skilled in the art.

As illustrated in FIG. 3A, each tine of tines 303 includes proximal portion 33 (e.g., which may correspond to first section S1) and distal portion 35 (e.g., which may correspond to second section S2 and third section S3). In some examples, free distal end 352 may include any suitable shape, such as, for example, a rounded shape as illustrated in FIG. 3A or an incisive shape. Proximal section 33-P is fixedly attached to base 301. Proximal section 33-P extends in a first direction d₁. In some examples, first direction d₁ may be substantially parallel to longitudinal axis 3. In some examples, first direction d₁ may be at an angle relative to longitudinal axis 3, such as for example, between about 0 degrees to about 5 degrees. Curved section 33-C may include a spring-biased pre-formed curvature. Curved section 33-C extends from proximal section 33-P laterally, outward from longitudinal axis 3 to distal portion 35. In some examples, curved section 33-C may include a single radius within a range from about 0.06 inch (1.524 mm) to about 0.08 inch (2.032 mm), such as about 0.067 inch±0.010 inch (1.7018 mm±0.254 mm). In some examples, proximal portion 33 may include more than one curved section.

Distal portion 35 may include a proximal section 35-P, a curved section 35-C, and a tip section 35-T. Proximal section 35-P may include a substantially straight segment extending in a second direction d₂ and along a relatively straight line (dashed line). In some examples, a length of proximal section 35-P may be within a range from about 0.075 inch (1.905 mm) to about 0.125 inch (3.175 mm), such as about 0.100 inch±0.005 inch (2.54 mm±0.127 mm). Proximal section 35-P may be oriented by curved section 33-C such that second direction d₂ is generally opposite first direction d₁ and the relatively straight line intersects longitudinal axis 3 at an acute angle θ. In some examples, angle θ is between about 30 degrees and about 60 degrees, such as about 45 degrees. Curved section 35-C may include a deformable pre-formed curvature. Curved section 35-C extends from proximal section 35-P (in direction d₂) back toward longitudinal axis 3 to tip section 35-T. In some examples, curved section 35-C, when un-deformed, is defined by a single radius within a range from about 0.045 inch (1.143 mm) to about 0.055 inch (1.397 mm), such as about 0.05 inch±0.010 inch. Tip section 35-T may include a substantially straight segment extending in a third direction d₃ from curved section 35-C to free distal end 352. In some examples, a length of each tip section 35-T may be within a range from about 0.055 inch (1.397 mm) to about 0.075 inch (1.905 mm), such as about 0.064 inch±0.005 inch (1.6256 mm±0.127 mm). Tip section 35-T is shown oriented by curved section 35-C, when un-deformed, to extend toward longitudinal axis 3, such that tip section 35-T and proximal section 35-P are shown enclosing an angle φ. In some examples, angle φ may be greater than or equal to about 90 degrees, such as in a range from about 90 degrees to about 120 degrees.

The shape (e.g., undeformed configuration) and width of each tine 303, and, in some examples, the super-elastic stiffness properties of nickel-titanium alloy, provide a sufficient spring force and structural stiffness for tines 303 to engage tissue for the fixation of IMD 20 at an implant site when deployed by delivery tool 430, as described in greater detail below. With reference to FIG. 3A, each tine 303 has a width “W” in a range from about 0.020 inch (0.508 mm) to about 0.035 inch (0.889 mm), such as about 0.031 inch (0.7874 mm). In some examples, a width of tines 303 may be selected to provide a radiopaque density that facilitates fluoroscopic visualization during and after the implant procedure.

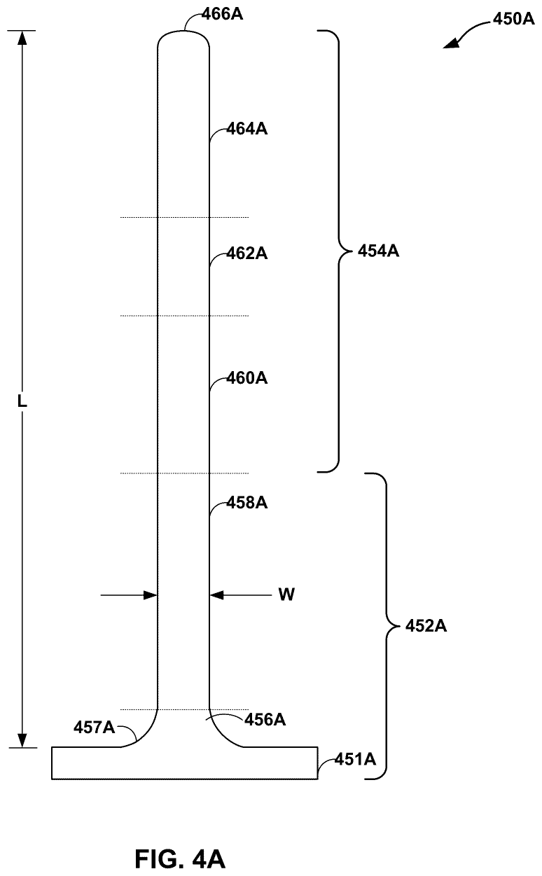

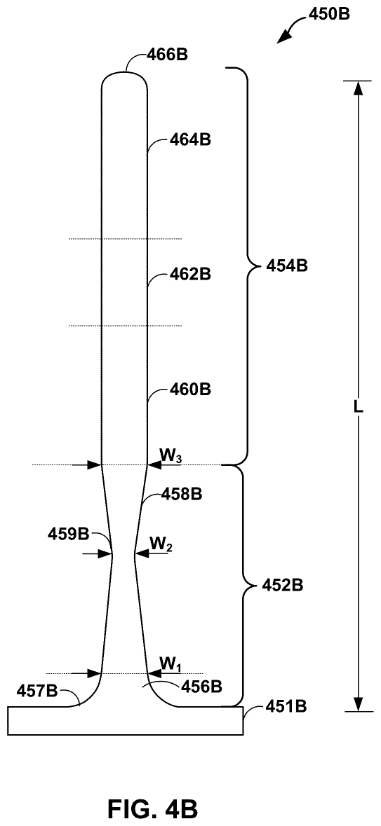

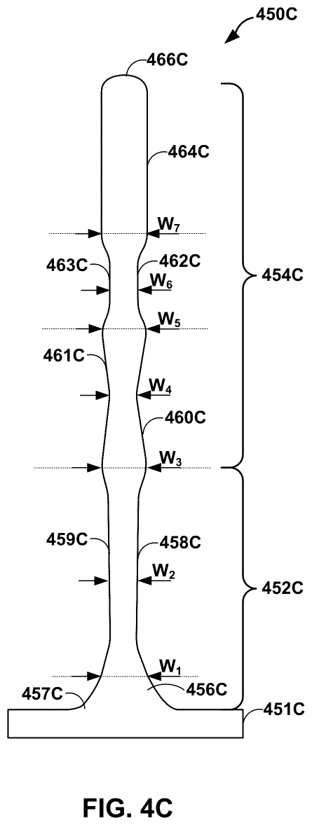

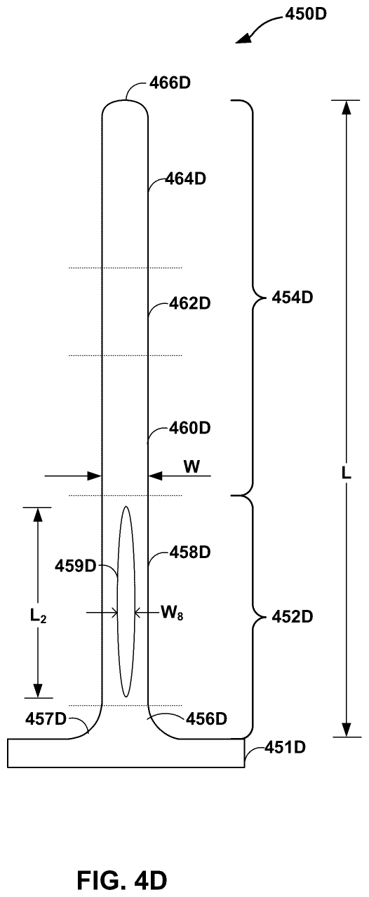

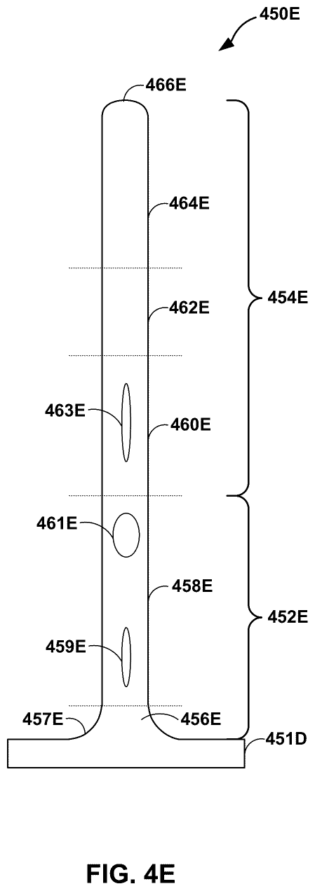

FIGS. 4A-4E are conceptual diagrams illustrating plan views of tines 450A-450E (collectively, tines 450) of the two-knuckle fixation component 300 illustrated in FIGS. 3A and 3B, prior to forming curves in the tine. Tines 450 may be the same as or substantially similar to tines 303 discussed above in reference to FIGS. 2A-3B, except for the differences described herein. For example, tines 450 may include a proximal portion 452 extending form base 451 and distal portion 454 extending from proximal portion 452. As indicated by the dashed lines indicating approximate boundaries of sections, proximal portion 452 may include a proximal section 456 and curved section 458, and distal portion 454 may include a proximal section 460, a curved section 462, and a tip section 464.

As illustrated in FIG. 4A, tine 450A may have a substantially constant width W (e.g., constant or nearly constant within the limits of common manufacturing tolerances) along the length L of tines 450A. For example, proximal portion 452A, including curved section 458A, and distal portion 454A, including proximal section 460A, a curved section 462A, and a tip section 464A, may have a substantially constant width. In some examples, proximal section 456A may include a fillet 457A extending from base 451A. Fillet 457A may reduce stress concentration at the junction of proximal section 456A and base 451A. The width W and length L of tine 450A may be the same as or substantially similar to tine 303 discussed above.

As illustrated in FIG. 4B, curved section 458B of tine 450B may include a tapered portion 459B. Tapered portion 459B includes a proximal width W₁, a medial (middle) width W₂, and a distal width W₃. In some examples, W₁ and W₃ may be the same or substantially similar, e.g., about 0.030 inch (0.762 mm), and W₂ may be about 0.025 inch (0.635 mm). Although illustrated as including three widths, in some examples, tapered portion 459B may include a plurality of tapers, each taper having a respective maximum width and respective minimum width. In some examples, tapered portion 459B may increase the flexibility of curved section 458B relative to an untampered curved section (e.g., curved section 458A illustrated in FIG. 4A). By increasing the flexibility of curved section 458B, tine 450B may have, after preforming curved section 458B as discussed above, an increased deflection stiffness compared to an untampered curved section. In some examples, distal portion 454B (e.g., proximal section 460B, curved section 462B, and tip 464B having free distal end 466B) may not include a tapered portion. By not including a tapered portion, tines 450B may have the same or substantially the same deployment stiffness compared to a tine having an untampered proximal portion (e.g., tine 450A). In this way, tines 450 may include one or more tapers to selectively control a deployment stiffness, a deflection stiffness, or both.

For example, as illustrated in FIG. 4C, tines 450C includes a plurality of tapers. Proximal section 456C may include fillet 457C extending from base 451C. Curved section 458C may extend from proximal section 456C and include a tapered portion 459C having a proximal width W₁, a medial width W₂, and a distal width W₃. Proximal section 460C of distal portion 454C may extend from curved section 458C and include a tapered portion 461C having a proximal width W₃, a medial width W₄, and a distal width W₅. Proximal section 460C of distal portion 454C may extend from curved section 458C and include a tapered portion 461C having a proximal width W₃, a medial width W₄, and a distal width W₅. Curved section 462C of distal portion 454C may extend from proximal section 462C and include a tapered portion 463C having a proximal width W₅, a medial width W₆, and a distal width W₇. As illustrated in FIG. 4C, each of tapers 459C, 461C, and 463C may include a unique shape. The unique shape of a respective tapered portion may be configured to have a target flexibility after preforming tine 450C, for example, such that tine 450C has a target deflection stiffness and deployment stiffness.

In some examples, rather than tapered portions, tines 450 may include cutouts, engravings, embossing, or other variations in the thickness of tines 450. For example, as illustrated in FIG. 4D, rather than a tapered portion, tine 450D includes cutout 458D having a width W₈ extending along a length L₂ of curved section 458C of proximal portion 452D. In some examples, cutout 458D may be configured to increase the flexibility of curved section 458D relative to an untampered curved section (e.g., curved section 458A illustrated in FIG. 4A). By increasing the flexibility of curved section 458D, tine 450D may have, after preforming curved section 458D as discussed above, an increased deflection stiffness compared to an untampered curved section.

As illustrated in FIG. 4E, tine 450E include a plurality of cutouts 459E, 451E, and 453E. Similarly as discussed above in reference to FIG. 4C, each of cutouts 459E, 451E, and 453E may include a unique shape and position configured to result in a target flexibility of tine 450E (e.g., after preforming tine 450C), such that tine 450C has a target deflection stiffness and a target deployment stiffness.

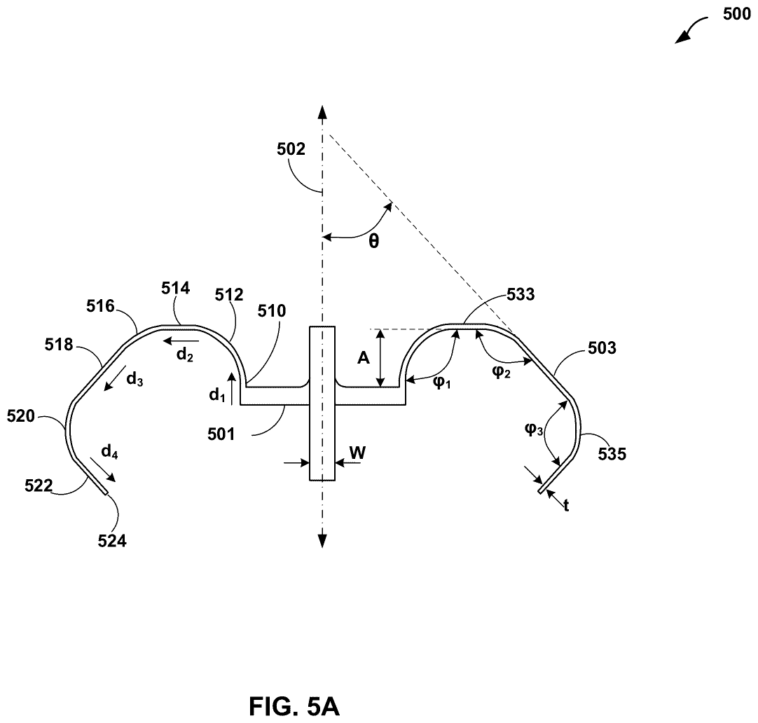

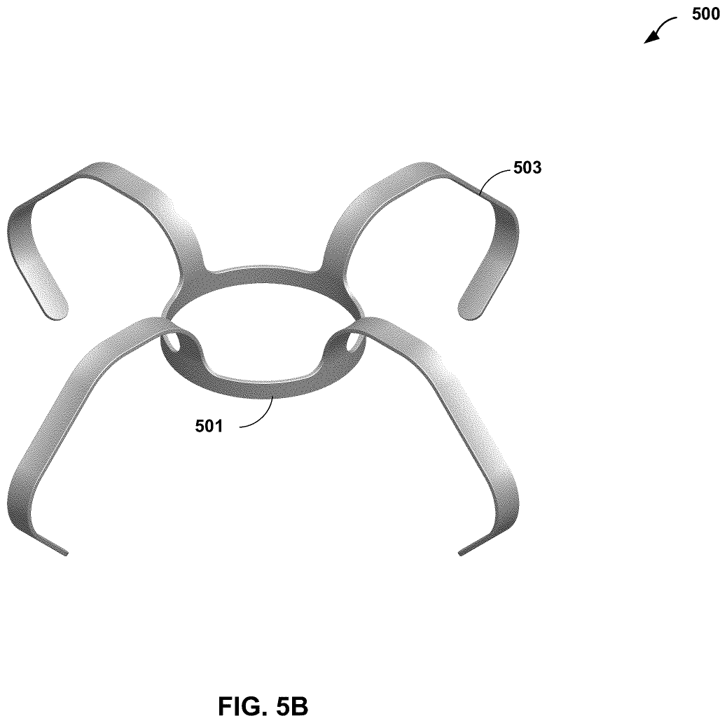

In some examples, the tines of a fixation component may include more than two curved sections to result in a target deflection stiffness and a target deployment stiffness. FIGS. 5A and 5B are conceptual diagrams illustrating an example three-knuckle fixation component 500. Three-knuckle fixation component 500 may be the same as or substantially similar to fixation component 30 and two-knuckle fixation component 300 discussed above in reference to FIGS. 2A-4E, except for the differences describe herein. For example, three-knuckle fixation component 500 includes a base 501 from which tines 503 extend and are spaced apart from one another around a perimeter of base 501. Base 501 may define a longitudinal axis 502 of three-knuckle fixation component 500, which may, in some examples, generally aligned along longitudinal axis 2 of IMD 20 (FIG. 2A).

As illustrated in FIG. 5A, each tine of tines 503 includes proximal portion 533 and distal portion 535 terminating in free distal end 552. Proximal section 510 of proximal portion 533 is fixedly attached to base 501. Proximal portion 533 may include a proximal section 510, a first curved section 512, a first straight section 514, and a second curved section 516, the first straight section 514 between the first and second curved sections 512 and 516. Each of proximal section 510, first curved section 512, first straight section 514, and second curved section 516 may be sized and shaped to enable tine 503 to have a target deflection stiffness and a target deployment stiffness.

Proximal section 510 extends in a first direction d₁. In some examples, first direction d₁ may be substantially parallel to longitudinal axis 502. In some examples, first direction d₁ may be an angle relative to longitudinal axis 502, such as for example, between about 0 degrees to about 5 degrees.

First curved section 512 may include a spring-biased pre-formed curvature. First curved section 512 extends from proximal section 510 laterally, outward from longitudinal axis 502 to first straight section 514. In some examples, first curved section 512 may include a single radius within a range from about 0.06 inch (1.524 mm) to about 0.08 inch (2.032 mm), such as 0.067 inch±0.010 inch (1.7018 mm±0.254 mm).

First straight section 514 may include a substantially straight segment extending in a second direction d₂ and along a relatively straight line to second curved section 516. In some examples, second direction d₂ may be perpendicular to longitudinal axis 502. In some examples, a length of first straight section 514 may be within a range from about 0.035 inch (0.889 mm) to about 0.045 inch (1.143 mm), such as about 0.04 inch (1.016 mm). First straight section 514 may be oriented by first curved section 512, when un-deformed, to extend away from longitudinal axis 502, such that proximal section 510 and first straight section 514 enclose an angle (p. In some examples, angle φ₁ may be within a range from about 75 degrees to about 105 degrees, such as about 90 degrees.

Second curved section 516 may include a spring-biased pre-formed curvature. Second curved section 516 extends from proximal section 510 laterally, outward from longitudinal axis 502 to second straight section 518 of distal portion 535. In some examples, second curved section 516 may include a single radius within a range from about 0.06 inch (1.524 mm) to about 0.08 inch (2.032 mm), such as 0.067 inch±0.010 inch (1.7018 mm±0.254 mm).

Distal portion 535 may include a second straight section 518, a third curved section 520, and a tip section 522. Second straight section 518 and/or third curved section 520 may be sized and shaped to enable tine 503 to have a target deflection stiffness, a target deployment stiffness, or both.

As discussed above, in some examples, the target deflection stiffness may be selected to enable tines 503 to deflect a predetermined amount to enable visualization of tines 503 under fluoroscopy. In some examples, the target deflection stiffness may be within a range from about 0.2 N to about 0.8 N, such as about 0.3 N to about 0.6 N. The deployment stiffness may include a measure of a force applied by tines 503 as tines 503 move from a deformed configuration to an undeformed configuration upon deployment of fixation component from distal opening of delivery tool such that free distal end 524 penetrates pectinate muscle PM. In some examples, the target deployment stiffness may be within a range from about 0.6N to about 1.2N.

Second straight section 518 may include a substantially straight segment extending in a third direction d₃ and along a relatively straight line (dashed line). In some examples, a length of second straight section 518 may be within a range from about 0.075 inch (1.905 mm) to 0.125 (3.175 mm) inch, such as 0.100 inch±0.005 inch (2.54 mm±0.127 mm). Second straight section 518 may be oriented by second curved section 516, when un-deformed, such that first straight section 514 and second straight section 518 enclose an angle φ₂. In some examples, angle φ₂ may be within a range from about 120 degrees to about 150 degrees, such as about 135 degrees.

Third curved section 520 may include a deformable pre-formed curvature. Third curved section 520 extends from second straight section 518 (in direction d₃) back toward longitudinal axis 502 to tip section 522. In some examples, third curved section 520, when un-deformed, is defined by a single radius within a range from about 0.045 inch (1.143 mm) to about 0.055 inch (1.397 mm), such as 0.05 inch±0.010 inch (1.27 mm±0.254 mm).

Tip section 522 may include a substantially straight segment extending in a fourth direction d₄ from third curved section 520 to free distal end 524. In some examples, a length of each tip section 522 may be within a range from about 0.045 inch (1.143 mm) to about 0.055 inch (1.397 mm), such as about 0.05 inch±0.010 inch (1.27 mm±0.254 mm). Tip section 522 is shown oriented by third curved section 520, when un-deformed, to extend toward longitudinal axis 502, such that tip section 522 and second straight section 518 are shown enclosing an angle φ₃. In some examples, angle φ₃ may be greater than or equal to about 90 degrees, such as in a range from about 90 degrees to about 120 degrees.

As discussed above in reference to FIGS. 2A-4E, the shaped configuration and width of each tine, e.g., tines 503, and, in some examples, the super-elastic stiffness properties of nickel-titanium alloy, enable each of tines 530 to produce a sufficient spring force and structural stiffness to engage tissue for the fixation of IMD 20 at an implant site when deployed by delivery tool 430, as described in greater detail below.

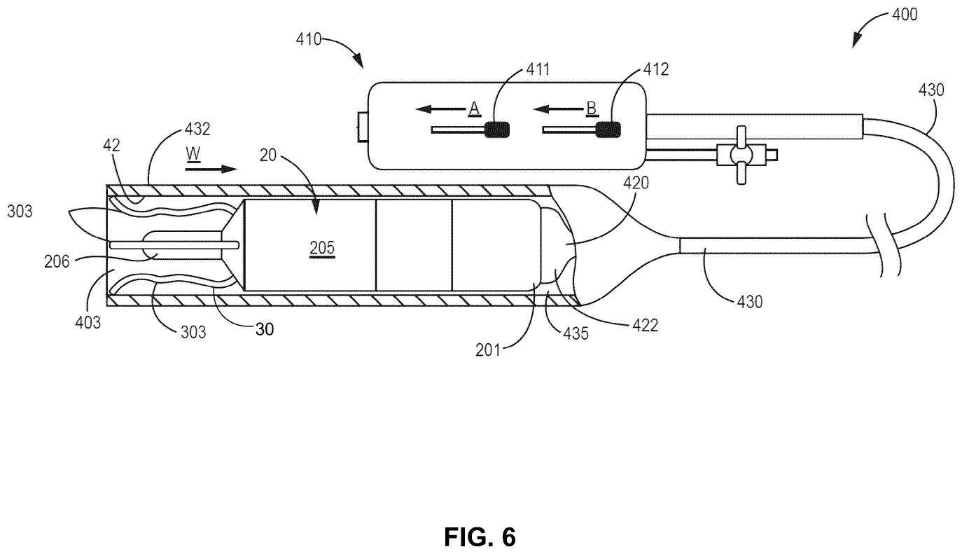

FIG. 6 is a conceptual diagram illustrating a plan view with a partial cut-away section of a medical device system 400 including a delivery tool 430 and an IMD 20. For purposes of illustration, the distal end of delivery tool 430 is enlarged relative to handle 410. Additionally, although medical device system 400 is described in reference to fixation component 30 describe in reference to FIGS. 2A and 2B, in other examples, medical device system 400 may include other fixation components, such as two-knuckle fixation component 300 describe in reference to FIGS. 3A and 3B or three-knuckle fixation component 500 illustrated in reference to FIGS. 5A and 5B.

During use, IMD 20 is loaded into delivery tool 430 for deployment to a target implant site (e.g. target implant site 102). Delivery tool 430 includes a handle 410, an elongate outer member 430, and an elongate inner member 420 that extends within lumen 435 of outer member 430. Inner member 420 includes a distal end 422, which is configured to engage IMD 20 by abutting proximal end 201 of housing 205 (e.g., as shown in the cut-away section). An entirety of IMD 20 may be loaded within tubular sidewall 432 that defines a distal portion of outer member lumen 435, for example, having been loaded therein by pulling IMD 20, with housing proximal end 201 leading, in through lumen distal opening 403. In some examples, an inner surface 42 of tubular sidewall 432 engages tines 303 of fixation component 30 as IMD 20 is loaded into lumen 435 to deform tines 303 (per arrow L of FIG. 3A) and then to hold each tine 303 of the loaded IMD 20 in a deformed configuration, e.g., a spring-loaded configuration.

With further reference to FIG. 6, a proximal end of outer member 430 is coupled to a control member 412 of handle 410 such that an entirety of outer member 430 is movable with respect to inner member 420, via control member 412, for example, so that an clinician may retract outer member 430, per arrow W, relative to IMD 20 and inner member 420, to deploy IMD 20 out through distal opening 403, after positioning medical device system 400 in proximity to a target implant site. The clinician may position medical device system 400 by advancing delivery tool 403 through a venous system of the patient, for example, from a femoral venous access site and up through the inferior vena cava IVC (FIG. 1). Delivery tool 430 may include articulating features to facilitate the navigation of the distal portion of delivery tool 430. For example, inner member 420 of delivery tool 430 may include a pull wire assembly (not shown) integrated therein and being coupled to another control member 411 of handle 410 that, when moved per arrow A, causes inner member 420 and outer member 430 to bend along distal portions thereof. A length of outer member 430, between handle 410 and distal opening 403, when outer member 430 is in the position shown in FIG. 6, may be between about 103 cm and about 107 cm, for example, to reach into the right atrium RA from the femoral access site. Suitable construction detail for a delivery tool like delivery tool 430 is described in co-pending and commonly assigned U.S. Pat. No. 9,526,522 to Wood et al., which is incorporated herein by reference in its entirety.

According to some methods, once the clinician has advanced medical device system 400 target implant site 102 (FIG. 1), so that distal opening 403 abuts pectinate muscle PM therein (FIG. 2B) at the target implant site, the clinician can move control member 412, per arrow B, to retract outer member 430 relative to IMD 20 and thereby release the spring loading of three-knuckle fixation component 500 so that tines 303 engage with pectinate muscle PM to secure IMD 20 at the implant site, as illustrated in FIG. 2B. However, it should be noted that, according to alternative embodiments and methods, delivery tool 430 may be configured so that an clinician can advance inner member 420 relative to outer member 430 to push IMD 20 out through distal opening 403 for deployment.

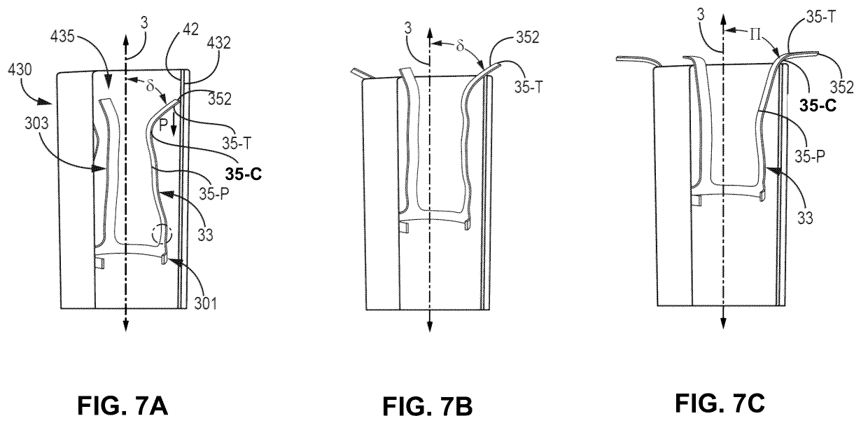

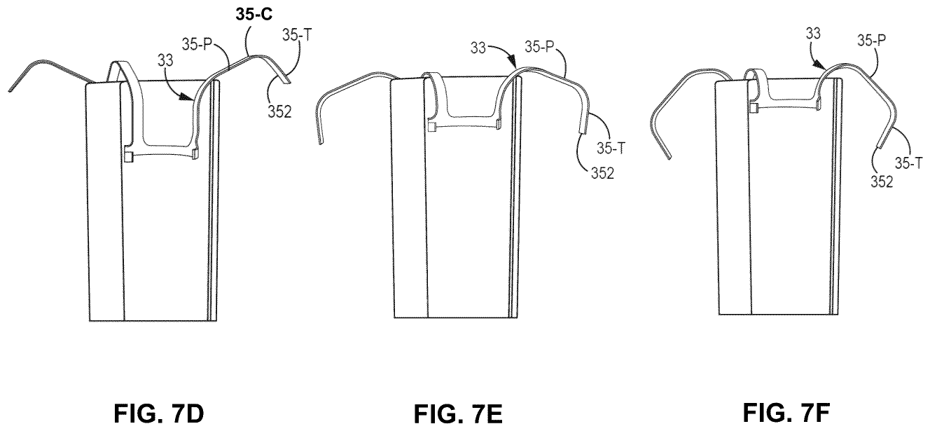

FIGS. 7A-7F are conceptual diagrams illustrating a sequence of positions and/or configurations corresponding to the release of above-described fixation components. Although illustrated in reference to fixation component 30 describe in reference to FIGS. 2A and 2B, in other examples, delivery tool 430 may be configured to release other fixation components, such as two-knuckle fixation component 300 describe in reference to FIGS. 3A and 3B or three-knuckle fixation component 500 illustrated in reference to FIGS. 5A and 5B. FIG. 7A is a conceptual diagram illustrating a spring loaded configuration of a fixation component within a lumen of a delivery tool. FIG. 7A illustrates a maximum deformation of tines 303 when held in the spring loaded configuration by the engagement of free distal end 352 with inner surface 42 of outer member tubular sidewall 432. In some examples, proximal portion 33 becomes relatively straightened. In some examples, a location of the maximum principle strain along each tine 303 is in relatively close proximity to base 301 (designated by dashed-line circle). In some examples, the length of the tip section 35-T and the associated angle φ, as describe above in reference to FIG. 3A, help to keep the deformed tines 303 from touching one another within lumen 435 and to prevent free distal ends 352 from being pulled proximally, per arrow P, when outer member 430 is retracted to release the spring loading of tines 303.

FIG. 7A further illustrates tip section 35-T extending away from axis 3 at an acute angle δ, which is preferably in a range from about 45 degrees to about 75 degrees for an initial release of the spring loading of each tine 303. For example, FIG. 7B is a conceptual diagram illustrating an initial release of the fixation component from the spring loaded configuration. Upon retraction of outer member 430, tip sections 35-T extend past the distal opening of sidewall 432.

FIG. 7C is a conceptual diagram illustrating movement of the tines causing initial penetration of the tissue after the initial release of the fixation component. For example, once free distal end 352 is released from engagement with inner surface 42 for deployment into tissue at the implant site, the spring force of proximal portion 33 and the pre-formed curvature of curved section 35-C cause tip section 35-T to immediately rotate away from axis 3 to an angle 7, which approaches 90 degrees, so that tip section 35-T is oriented approximately normal to axis 3 for initial penetration of pectinate muscle PM. Thus each tine free distal end 352 is deployed in a direction toward pectinate muscle PM that ultimately prevents tines 303 from perforating the underlying visceral pericardium VP (reference FIG. 2B).

FIGS. 7D-7F illustrate the subsequent movement of tines 303, being driven by the release of proximal portion 33 from the spring loaded configuration. FIG. 7D is a conceptual diagram illustrating further movement of the fixation component as the portions of the tines between the distal most and next proximal curves reaches the distal end of the delivery tool. As each tine of tines 303 moves from the position illustrated in FIG. 7C to the position illustrated in FIG. 7D, free distal end 352 may travel substantially transverse to longitudinal axis 3, thereby penetrating additional tissue. FIG. 7E is a conceptual diagram illustrating further movement fixation component as the proximal curve travels past the distal end of the delivery tool. FIG. 7F is a conceptual diagram illustrating final configuration of fixation component movement, subsequent to movement. As illustrated in FIGS. 7D-7F, the release of proximal portion 33 may cause free distal end 352 to curl back toward longitudinal axis 3, such that, after penetrating through pectinate muscle PM at a first location P1, tip section 35-T may penetrate back through pectinate muscle PM in an opposite direction at a second location P2, so that IMD 20 may be securely fixed at the implant site, as illustrated in FIG. 2B.

The configuration of distal portion 35, including, for example, the length of proximal section 35-P and tip section 35-T, and the pre-formed curvature of curved section 35-C, provide a structural stiffness and reach to each tine 303 that is sufficient for deformation and subsequent penetration of free distal end 352 through pectinate muscle PM, as shown in FIG. 2B, but is not sufficient for penetration through visceral pericardium VP. Even if the clinician ends up advancing medical device system 400 into target implant site 102 so that distal opening 403 of delivery tool 430 abuts visceral pericardium VP, between folds of pectinate muscle PM, free distal end 352, according to this configuration of tines 303, is not backed-up by sufficient stiffness to penetrate through visceral pericardium VP, so tip section 35-T of tine distal portion 35 is redirected, laterally, toward pectinate muscle PM.

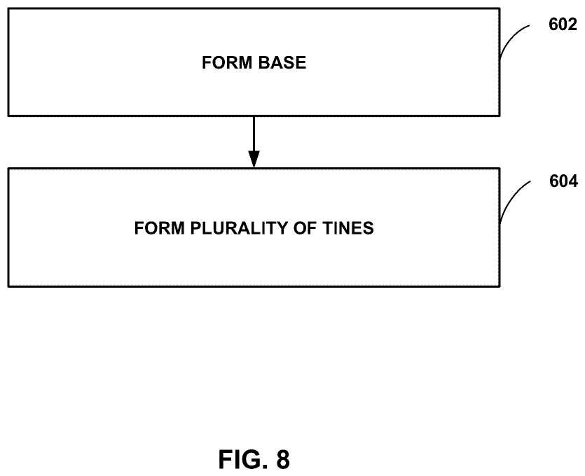

The fixation components described herein may be manufactured using any suitable technique. FIG. 8 is a flow diagram illustrating an example method of manufacturing a three-knuckle fixation component 500. Although the technique illustrated in FIG. 8 is described in reference to three-knuckle fixation component 500 illustrated in reference to FIGS. 5A and 5B, the technique may be used to manufacture other fixation component, such as fixation component 30 describe in reference to FIGS. 2A, 2B, 6, and 7A-7F, and two-knuckle fixation component 300 describe in reference to FIGS. 3A and 3B. Additionally, fixation component 30 and/or two-knuckle fixation component 300 may be manufactured using other techniques.

The technique illustrated in FIG. 8 includes forming base 501 defining longitudinal axis 502 of fixation component 500. In some examples, forming based 501 may include cutting a tube, such as a metal tube, a nickel titanium alloy tube, or a stainless steel tube, to define base 501. Forming base 501 may include pre-processing or post-processing steps, such as abrading, coating, heat treating, or polishing a substrate defining base 501.

The technique illustrated in FIG. 8 also includes forming tines 503 extending from base 501 and being spaced apart from one another. In some examples, base 501 and tines 503 may be integrally formed. For example, base 501 and tines 503 may be integrally formed from a tube, such as a metal tube, a nickel titanium alloy tube, or a stainless steel tube. In some examples, forming base 501 and tines 503 from a single tube may include removing material from the single tube to define base 501 and tines 503. In some examples, removing material from the single tube may include one or more of machining, chemical etching, laser etching, stamping, or water cutting. In some examples, forming tines 503 may include forming one or more tapers on one or more tines of the plurality of tines. For example, forming one or more tapers may include any other above techniques to remove material from the single tube. In some examples, one or more tapers may be formed while removing material form the single tube.

In some examples, forming tines 503 may include bending each tine of tines 503 to define first curved section 512, second curved section 516, and third curved section 520. In some examples, each curve and/or each tine of tines 503 may be bent individually or bend simultaneously, e.g., by use of a jig configured to bend one or more curves on one or more of tines 503. After bending (and holding in the bent configuration) tines 503, forming tines 503 also may include heat treating the bent tines 503 to cause the plurality of tines to hold the bent configuration. For example, heat treating the bent tines 503 may cause a microstructure of the material of tines 503 to assume a configuration such that a resting state of tines 503 (e.g., without application of an external force) is the bend configuration.

The following clauses illustrate example subject matter of the present disclosure.

Clause 1. A fixation component for an implantable medical device (IMD), comprising: a base defining a longitudinal axis of the fixation component, wherein the base is fixedly attached to the IMD having a proximal end and a distal end aligned along the longitudinal axis; and a plurality of tines extending from the base and being spaced apart from one another, each tine of the plurality of tines comprising: proximal portion comprising: a proximal section fixedly attached to the base and extending from the base in a first direction; a first curved section defining a first deformable pre-formed curvature and extending from the proximal section laterally, outward from the longitudinal axis; a first straight section extending from the first curved section laterally, outward from the longitudinal axis in a second direction; and a second curved section defining a second deformable pre-formed curvature and extending from the first straight section laterally, outward from the longitudinal axis; and a distal portion comprising: a second straight section extending from the second curved section in a third direction oriented generally opposite the first direction; a third curved section defining a third deformable pre-formed curvature and extending from the second straight section; and tip section extending from the third curved section toward the longitudinal axis and terminating in a free distal end.

Clause 2. The fixation component of clause 1, wherein the proximal portion is configured to have a deflection stiffness of less than about 0.6 Newtons (N).

Clause 3. The fixation component of clause 1 or 2, wherein the proximal portion is configured to have a deployment stiffness of more than about 0.6 N.

Clause 4. The fixation component of any one of clauses 1 through 3, wherein the first curved section, when in an undeformed configuration, is defined by a radius of the first deformable pre-formed curvature within a range from about 1.524 millimeters (mm) to about 2.032 mm.

Clause 5. The fixation component of any one of clauses 1 through 4, wherein the second curved section, when in an undeformed configuration, is defined by a radius of the second deformable pre-formed curvature within a range from 1.524 mm to about 2.032 mm.

Clause 6. The fixation component of any one of clauses 1 through 5, wherein the third curved section, when in an undeformed configuration, is defined by a radius of the third deformable pre-formed curvature within a range from about 1.143 mm to about 1.397 mm.

Clause 7. The fixation component of any one of clauses 1 through 6, wherein the first straight section has a length within a range from about 0.889 mm to about 1.143 mm.

Clause 8. The fixation component of any one of clauses 1 through 7, wherein the second straight section has a length within a range from about 1.905 mm to about 3.175 mm.

Clause 9. The fixation component of any one of clauses 1 through 8, wherein the first straight section and the second straight section enclose an angle within a range from about 120 degrees to about 150 degrees.

Clause 10. The fixation component of any one of clauses 1 through 9, wherein the tip section has a length within a range from about 1.143 mm to about 1.397 mm.

Clause 11. The fixation component of any one of clauses 1 through 10, wherein the third curve section, when un-deformed, orients the tip section to enclose with the second straight section an angle within a range from about 90 degrees to about 120 degrees.

Clause 12. The fixation component of any one of clauses 1 through 11, wherein at least one of the first curve section or the second curve section comprises a tapered portion comprising a width of less than about 0.762 millimeters.

Clause 13. The fixation component of any one of clauses 1 through 12, wherein at least one of the first straight section, second straight section, or tip section comprises a tapered portion comprising a width of less than about 0.762 millimeters.

Clause 14. The fixation component of any one of clauses 1 through 13, wherein the tapered portion comprises a proximal portion having a width of about 0.762 mm, a medial portion having a width of about 0.635 mm, and a distal portion having width of about 0.762 mm.

Clause 15. The fixation component of any one of clauses 1 through 14, wherein the tapered portion comprises a change in a width of the tine of at least about 0.127 mm.

Clause 16. The fixation component of any one of clauses 1 through 15, wherein the tapered portion comprises a cutout, and wherein the width comprises a width of the tine less a widest portion of the cutout.

Clause 17. A fixation component for an implantable medical device (IMD), comprising: a base defining a longitudinal axis of the fixation component, wherein the base is fixedly attached to the IMD having a proximal end and a distal end aligned along the longitudinal axis; and a plurality of tines extending from the base and being spaced apart from one another, each tine of the plurality of tines comprising: a proximal portion comprising: a proximal section fixedly attached to the base and extending in a first direction generally parallel to the longitudinal axis; and a first curved section extending from the proximal section laterally, outward from the longitudinal axis, wherein the curved section is configured to provide a deflection stiffness of less than about 0.6 Newtons; a distal portion comprising: a second proximal section extending from the first curved section in a second direction oriented generally opposite the first direction; a second curved section having a deformable pre-formed curve and extending from the second proximal section; and tip section extending from the second curved section toward the longitudinal axis and terminating in a free distal end.

Clause 18. The fixation component of clause 17, wherein the first curved comprises a width of equal to or less than 0.635 millimeters.

Clause 19. The fixation component of clause 17 or 18, wherein at least a portion of a respective tine of the plurality of tines comprises a taper.

Clause 20. The fixation component of clause 19, wherein the portion of the respective tine comprises the second proximal section.

Clause 21. The fixation component of clause 19 or 20, wherein the taper comprises proximal portion having a width of about 0.762 mm, a medial portion having a width of about 0.635 mm, and a distal portion having width of about 0.762 mm.

Clause 22. The fixation component of any one of clauses 19 through 21, wherein the taper comprises a change in a width of the tine of at least about 0.127 mm.

Clause 23. The fixation component of any one of clauses 17 through 22, wherein the first curved section is defined by a single radius, the radius being between about 1.524 mm and about 2.032 mm.

Clause 24. The fixation component of any one of clauses 17 through 23, wherein the first curved section comprises a first deformable pre-formed curved section and a second deformable pre-formed curved section.

Clause 25. The fixation component of clause 24, wherein the first curved section further comprises a straight section extending between the first deformable pre-formed curved section and the second deformable pre-formed curved section.

Clause 26. The fixation component of clause 24 or 25, wherein the deflection stiffness of the first curved section is defined by a first radius of the first deformable pre-formed curvature when in an undeformed configuration, a second radius of the second deformable pre-formed curvature when in an undeformed configuration, and a length of the straight section extending therebetween.

Clause 27. An implantable medical device (IMD) comprising: a housing extending along a longitudinal axis from a proximal end to a distal end; an electrode mounted in proximity to the distal end of the housing; and a fixation component comprising a base in proximity to the distal end of the housing and a plurality of tines fixedly attached spaced from one another around a perimeter of the distal end of the housing, each tine of the plurality of tines comprising: a proximal portion comprising: a proximal section fixedly attached to the base and extending from the base in a first direction; a first curved section defining a first deformable pre-formed curvature and extending from the proximal section laterally, outward from the longitudinal axis; a first straight section extending from the first curved section laterally, outward from the longitudinal axis in a second direction; and a second curved section defining a second deformable pre-formed curvature and extending from the first straight section laterally, outward from the longitudinal axis; and a distal portion comprising: a second straight section extending from the second curved section in a third direction oriented generally opposite the first direction; a third curved section defining a third deformable pre-formed curvature and extending from the second straight section; and tip section extending from the third curved section toward the longitudinal axis and terminating in a free distal end.

Clause 28. The IMD of clause 27, wherein the proximal portion is configured to have a deflection stiffness of at least less than 0.6 N.

Clause 29. The IMD of clause 27 or 28, wherein the proximal portion is configured to have a deployment stiffness of at least more than 0.6 N.

Clause 30. A medical device system comprising: an implantable medical device (IMD) comprising: a housing extending along a longitudinal axis from a proximal end to a distal end; an electrode mounted in proximity to the distal end of the housing; and a fixation component comprising a base in proximity to the distal end of the housing and a plurality of tines fixedly attached spaced from one another around a perimeter of the distal end of the housing; and a delivery tool comprising a tubular sidewall that defines a lumen into which the IMD may be loaded, wherein the lumen having a distal opening through which the IMD may be deployed, wherein each tine of the plurality of tines comprises: proximal portion comprising: a proximal section fixedly attached to the base and extending from the base in a first direction; a first curved section defining a first deformable pre-formed curvature and extending from the proximal section laterally, outward from the longitudinal axis; a first straight section extending from the first curved section laterally, outward from the longitudinal axis in a second direction; and a second curved section defining a second deformable pre-formed curvature and extending from the first straight section laterally, outward from the longitudinal axis; and distal portion comprising: a second straight section extending from the second curved section in a third direction oriented generally opposite the first direction; a third curved section defining a third deformable pre-formed curvature and extending from the second straight section; and tip section extending from the third curved section toward the longitudinal axis and terminating in a free distal end.

Clause 31. The medical device system of clause 30, wherein the proximal portion is configured to have a deflection stiffness of at least less than 0.6 N.

Clause 32. The medical device system of clause 30 or 31, wherein the proximal portion is configured to have a deployment stiffness of at least more than 0.6 N.

Clause 33. The medical device system of any one of clauses 30 through 32, wherein, when the IMD is loaded within the lumen of the delivery tool, the free distal end of each tine of the fixation component engages an inner surface of the tubular sidewall in proximity to the distal opening of the delivery tool to hold at least one of the first curved section, the second curved section, or the third curved section of each tine of the plurality of tines in a spring loaded configuration in which: each tip section extends away from the longitudinal axis at an acute angle in a range from about 45 degrees to about 75 degrees for deployment of the corresponding free distal end out from the distal opening; and upon deployment, each tip section rotates away from the longitudinal axis to approach an angle of about 90 degrees relative to the longitudinal axis in response to an initial release of the spring loaded configuration of at least one of the first curved section, the second curved section, or the third curved section of each tine of the plurality of tines.

Clause 34. A method of forming a fixation component for an IMD comprising: forming a base defining a longitudinal axis of the fixation component; and forming a plurality of tines extending from the base and being spaced apart from one another, each tine of the plurality of tines comprising: a proximal portion comprising: a proximal section fixedly attached to the base and extending from the base in a first direction; a first curved section defining a first deformable pre-formed curvature and extending from the proximal section laterally, outward from the longitudinal axis; a first straight section extending from the first curved section laterally, outward from the longitudinal axis in a second direction; and a second curved section defining a second deformable pre-formed curvature and extending from the first straight section laterally, outward from the longitudinal axis; and a distal portion comprising: a second straight section extending from the second curved section in a third direction oriented generally opposite the first direction; a third curved section defining a third deformable pre-formed curvature and extending from the second straight section; and tip section extending from the third curved section toward the longitudinal axis and terminating in a free distal end.

Clause 35. The method of clause 34, wherein the base and the plurality of tines are integrally formed from a tube by removing material from the tube to define the base and the plurality of tines.

Clause 36. The method of clause 34 or 35, wherein forming the plurality of tines comprises: bending each tine of the plurality of tines to define the first curved section, the second curved section, and the third curved section; and heat treating the bent plurality of tines to cause the plurality of tines to hold the bent configuration.

Clause 37. The method of any one of clauses 34 through 36, wherein the method comprises forming one or more tapers on one or more tines of the plurality of tines.

Various examples of the disclosure have been described. Any combination of the described systems, operations, or functions is contemplated. These and other examples are within the scope of the following claims.

1: A fixation component for an implantable medical device (IMD), comprising:
a base defining a longitudinal axis of the fixation component, wherein the base is fixedly attached to the IMD having a proximal end and a distal end aligned along the longitudinal axis; and
a plurality of tines extending from the base and being spaced apart from one another, each tine of the plurality of tines comprising:
a proximal portion comprising:
a proximal section fixedly attached to the base and extending from the base in a first direction;
a first curved section defining a first deformable pre-formed curvature and extending from the proximal section;
a first straight section extending from the first curved section in a second direction; and
a second curved section defining a second deformable pre-formed curvature and extending from the first straight section, wherein at least one of the first curved section or the second curved section comprises a proximal tapered portion; and
a distal portion comprising:
a tip section terminating in a free distal end, wherein the tip section comprises a distal tapered portion.

2-3. (canceled)

4: The fixation component of claim 1, wherein the first curved section, when in an undeformed configuration, is defined by a radius of the first deformable pre-formed curvature within a range from about 1.524 millimeters (mm) to about 2.032 mm.

5: The fixation component of claim 1, wherein the second curved section, when in an undeformed configuration, is defined by a radius of the second deformable pre-formed curvature within a range from 1.524 mm to about 2.032 mm.

6: The fixation component of claim 1, wherein the third curved section, when in an undeformed configuration, is defined by a radius of the third deformable pre-formed curvature within a range from about 1.143 mm to about 1.397 mm.

7: The fixation component of claim 1, wherein the first straight section has a length within a range from about 0.889 mm to about 1.143 mm.

8: The fixation component of claim 1, wherein the second straight section has a length within a range from about 1.905 mm to about 3.175 mm.

9: The fixation component of claim 1, wherein the first straight section and the second straight section enclose an angle within a range from about 120 degrees to about 150 degrees.

10: The fixation component of claim 1, wherein the tip section has a length within a range from about 1.143 mm to about 1.397 mm.

11: The fixation component of claim 1, wherein the third curve section, when un-deformed, orients the tip section to enclose with the second straight section an angle within a range from about 90 degrees to about 120 degrees.

12: The fixation component of claim 1, wherein the proximal tapered portion comprises a width of less than about 0.762 millimeters.

13: The fixation component of claim 1, wherein the distal tapered portion comprises a width of less than about 0.762 millimeters.

14: The fixation component of claim 1, wherein at least one of the proximal tapered portion or the distal tapered portion comprises a proximal portion having a width of about 0.762 mm, a medial portion having a width of about 0.635 mm, and a distal portion having width of about 0.762 mm.

15: The fixation component of claim 1, wherein at least one of the proximal tapered portion or the distal tapered portion comprises a change in a width of the tine of at least about 0.127 mm.

16: The fixation component of claim 1, wherein at least one of the proximal tapered portion or the distal tapered portion comprises a cutout, and wherein the width comprises a width of the tine less a widest portion of the cutout.

17-20. (canceled)

21: An implantable medical device (IMD) comprising:
a housing extending along a longitudinal axis from a proximal end to a distal end;
an electrode mounted in proximity to the distal end of the housing; and
a fixation component comprising a base in proximity to the distal end of the housing and a plurality of tines fixedly attached spaced from one another around a perimeter of the distal end of the housing, each tine of the plurality of tines comprising, when in an undeformed configuration:
a proximal portion comprising:
a proximal section fixedly attached to the base and extending from the base in a first direction;
a first curved section defining a first deformable pre-formed curvature and extending from the proximal section;
a first straight section extending from the first curved section in a second direction; and
a second curved section defining a second deformable pre-formed curvature and extending from the first straight, wherein at least one of the first curve section or the second curve section comprises a proximal tapered portion; and
a distal portion comprising:
a tip section terminating in a free distal end, wherein the tip section comprises a distal tapered portion.

22: A medical device system comprising:
an implantable medical device (IMD) comprising:
a housing extending along a longitudinal axis from a proximal end to a distal end;
an electrode mounted in proximity to the distal end of the housing; and
a fixation component comprising a base in proximity to the distal end of the housing and a plurality of tines fixedly attached spaced from one another around a perimeter of the distal end of the housing; and
a delivery tool comprising a tubular sidewall that defines a lumen into which the IMD may be loaded, wherein the lumen having a distal opening through which the IMD may be deployed, and wherein each tine of the plurality of tines comprises, when in an undeformed configuration:
a proximal portion comprising:
a proximal section fixedly attached to the base and extending from the base in a first direction;
a first curved section defining a first deformable pre-formed curvature and extending from the proximal section;
a first straight section extending from the first curved section in a second direction; and
a second curved section defining a second deformable pre-formed curvature and extending from the first straight, wherein at least one of the first curve section or the second curve section comprises a proximal tapered portion; and
a distal portion comprising:
a tip section terminating in a free distal end, wherein the tip section comprises a distal tapered portion.

23: The fixation component of claim 1, wherein the distal portion further comprises:
a second straight section extending from the second curved section in a third direction; and
a third curved section defining a third deformable pre-formed curvature and extending from the second straight section, wherein the tip section extends from the third curved section.

24: The IMD of claim 21, wherein the distal portion further comprises:
a second straight section extending from the second curved section in a third direction; and
a third curved section defining a third deformable pre-formed curvature and extending from the second straight section, wherein the tip section extends from the third curved section.

25: The medical device system of claim 22, wherein the distal portion further comprises:
a second straight section extending from the second curved section in a third direction; and
a third curved section defining a third deformable pre-formed curvature and extending from the second straight section, wherein the tip section extends from the third curved section.
